(19) United States
(12) Patent Application Publication
(10) Pub. No.: US 2007/0179457 A1
(43) Pub. Date: Aug. 2, 2007

(54) DEVICE AND METHOD FOR THE ADMINISTRATION OF EYE DROPS

(76) Inventors: Willet F. Whitmore, Longboat Key, FL (US); Charles R. Putrino II, Nokomis, FL (US); Stephen E. Brauner, Bradenton, FL (US); Winston E. Barzell, Sarasota, FL (US)

Correspondence Address: PAUL D. BIANCO: FLEIT, KAIN, GIBBONS,;GUTMAN, BONGINI, & BIANCO P.L., 21355 EAST DIXIE HIGHWAY, SUITE 115, MIAMI, FL, 33180, US

(21) Appl. No.: 11/561,872

(22) Filed: Nov. 20, 2006

Related U.S. Application Data
(60) Provisional application No. 60/737,767, filed on Nov. 18, 2005.

(51) Int. Cl. A61M 35/00

(52) U.S. Cl. 604/298; 604/295

(57) ABSTRACT

An eye drop applicator system includes a barrel-shaped body, a stem, and a fluid directing portion. The stem is adapted to retain a fluid reservoir for providing eye drop fluid. A method for administering eye drops to an eye includes demountably coupling a fluid reservoir to a fluid directing portion associated with a barrel-shaped body so that a tip of the fluid reservoir communicates with a conduit in the fluid directing portion; rotating the barrel-shaped body against a lower eyelid to retract the eyelid and expose a cul-de-sac of the eye; and transmitting fluid from the fluid reservoir to the cul-de-sac through an opening in the fluid directing portion.

### CROSS-REFERENCE TO RELATED APPLICATION

The benefits of Provisional Application No. 60/737,767 filed Nov. 18, 2005 and entitled “Device and Method for the Administration of Eye Drops” are claimed under 35 U.S.C. §119(e) and the entire contents of this application are expressly incorporated herein by reference thereto.

### FIELD OF THE INVENTION

The invention relates to a device and method for administering eye drops. In particular, the invention relates to a device for exposing the cul-de-sac of the eye while one or more drops are applied.

### BACKGROUND OF THE INVENTION

Eye drops typically have been administered using a common manual bulb-operated pipette or a small squeeze bottle with a dropper tip. The size and tip configuration of these common “droppers” permits accurate self-administration of a single drop onto the eye assuming the user has good vision and sufficient manual dexterity. However, this is often not the case, and drops are frequently misplaced onto the eyelashes, facial skin near the eye, or even miss the face completely. Consequently, inconsistent patient compliance with the use of eye drop medications is a widely recognized problem.

A variety of devices are known for the placement of eye drops. For example, U.S. Pat. Nos. 5,154,710 and 5,154,711 to Williams are directed to an ophthalmic device that includes a plastic cup with a port adapted to receive a liquid dispenser containing a medicament. The dispenser is demountably retained by the cup. In use, prior to locating the device over the eye, a drop dispenser is mounted in a first port. The device is positioned so that the lower lid of the eye is drawn downward slightly to expose more of the eye ball and facilitate the entry of medicament into the eye. Such a device is commercially available as the Autodrop™ Eye Drop Guide by Owen Mumford Ltd (Oxford, England).

Another eye drop applicator attachment is disclosed in U.S. Pat. No. 5,037,406 to Smith et al. The applicator attachment is provided with a hollow cylindrical body, and an eyecup integral with the body at one end. The applicator attachment may be attached to a squeeze bottle. Such a device is commercially available as the SUREDROP™ eye drop guide by Suredrop, Inc. (Atlanta, Ga.).

Other commercially-available aids for placing drops of ophthalmic solution include the Eye Ease™ Eye Drop Guide by Maddak Inc. (Pequannock, N.J.), the Eye Drop Delivery Aid distributed by Upsher-Smith Laboratories, Inc. (Minneapolis, Minn.), the Xal-Ease™ delivery aid available from Pharmacia Corp. (Kalamazoo, Mich.), and the EZY-DROP GUIDE™.

Typical devices are configured to connect to a multi-dose dropper bottle and rely on the bony orbit of the eye (as well as the nose in some cases) to guide a dropper tip into a central position in front of the eye. Some devices assist in holding the eye open by frictionally restricting eyelid movement. Typically, the devices rely on tilting the user's head backward so that gravity will deliver the drop onto the eye. Alternatively, the devices assist in providing a forceful squirt for projecting a stream of multiple drops although this method generally is not desirable because of wasted medication and discomfort.

Many eye care professionals recommend that the inferior cul-de-sac of the eye be opened to receive a drop. To this end, the lower lid may be pulled downward to expose the potential space between the lower lid and the eyeball. This maneuver results in a shallow, crescent-shaped cavity or so-called cul-de-sac for receiving a drop. Some commercially available devices have the drawback that the user must open the cul-de-sac with a finger before placing the device, then place the device while attempting to maintain this retraction, and then administer the drop. Such a technique for placing drops is cumbersome. Although some of the commercially available devices are designed to be pressed onto the peri-orbital skin to open the cul-de-sac, the contact area may be too broad and the device operation may be inefficient.

Typically, the dropper tip is centered over the eye due to its fixation point in the eye drop applicator device to which it is coupled. The dropper tip also is elevated with respect to the eye and remote from the cul-de-sac due to the coupling position. Because of these features, a user must tilt his or her head backward significantly to get the drop to fall into the cul-de-sac or onto the eye. The remotely-disposed dropper tip combined with the required head tilt also adversely impact the user's ability to use a visual aid such as a mirror during the eye drop application process resulting in loss of accuracy and unreliable delivery of the drug.

Another problem with placing eye drops is sterility. Multi-dose bottles are at risk for contamination by exposure of the tip and the reflex suction of the bottle to the air or even to body fluids if the tip comes in contact with the patient. Until very recently, pharmaceutical companies have addressed this problem by including preservatives and/or antiseptics in eye drops. Alternatively, they have packaged eye drop medications in inexpensive single dose (unit dose) dropper squeeze bottles that are discarded after one dose is taken. Also, recently Pfizer Corp. has introduced a new multi-dose dispenser called Visine® Pure Tears that releases single drop doses from a dropper tip bottle that greatly reduces the risk of reservoir contamination. The device uses a spring valve mechanism and an antibacterial silver coil to reduce the risk of contamination.

To avoid any contribution to the risk of infection, dropper assist devices (which are typically non-sterile devices) are designed so that the eye drops do not contact the device before reaching the eye. In addition, the assist devices do not contact the mucous membranes of the eye. In order to facilitate this, the devices are often hemispherical in shape (eye cups) and larger than the bony structures of the orbit to prevent possible contact. Consequently, by necessity, they largely obscure the dropper tip and the eye, and this creates uncertainty during use. Also, these dropper assist devices are not designed for the unit dose market with its tiny, pre-filled and sealed sterile disposable containers for unit dosing.

Many prior art eye drop assist products attempt to reduce the need for good manual dexterity and direct viewing of the process by relying on essentially passive anatomic orientation of the device to the structures surrounding the eye. These assist devices require tilting the head back far enough so that the centered but somewhat remote dropper tip is positioned to permit a drop to fall onto the eye.

Many prior art assist devices are bulky, obscure the eye so that visual feedback is limited, must be kept clean or washed between uses, and only work with larger multi-dose bottles. Such multi-dose bottles are at risk for contamination and are more difficult to control in terms of dose delivery, often producing multiple unintended drops with a light squeeze.

There remains a need for improved devices and methods for delivering eye drops.

### SUMMARY OF THE INVENTION

The invention relates to an eye drop applicator system including a barrel-shaped body, a stem, and a fluid directing portion, wherein the stem is adapted to retain a fluid reservoir for providing eye drop fluid. The barrel-shaped body and stem may be integrally formed. In some embodiments, the barrel-shaped body and fluid directing portion may be integrally formed, while in other embodiments the fluid directing portion is demountably coupled to the barrel-shaped body. The barrel-shaped body may have a keyway that receives a like-shaped protruding region on the fluid directing portion. The fluid directing portion may be sized to extend along a longitudinal axis of the barrel-shaped body beyond the barrel-shaped body to form a free end of the eye drop applicator system. The free end may be formed as a bulbous portion.

The fluid directing portion may have a first opening for receiving fluid from the fluid reservoir and a second opening for dispensing fluid from the fluid reservoir. Also, the fluid directing portion may be a conduit extending from the first opening to the second opening, with the conduit optionally having first and second sections disposed substantially perpendicular to one another. The fluid directing portion may have a drip release portion proximate an opening thereof.

The barrel-shaped body may have texturing. In some embodiments, the barrel-shaped body may be disposed along a longitudinal axis and the texturing may include ribs disposed substantially parallel to the longitudinal axis.

The stem may be bifurcated and may extend substantially parallel to a longitudinal axis of the barrel-shaped body. In addition, the stem may include a clamping portion for releasably retaining the fluid reservoir. In some embodiments, the stem may include a ring-shaped portion for receiving a neck of the fluid reservoir. Also in some embodiments, the ring-shaped portion may be threaded for threadably receiving the neck of the fluid reservoir.

At least one selected from the group consisting of the barrel-shaped body and the fluid directing portion may be latex-free.

The stem may include a coupling portion for receiving a fluid reservoir with a tip so that the tip is oriented transverse to a longitudinal axis of the barrel-shaped body. In some embodiments, the tip may be oriented substantially perpendicular to the longitudinal axis of the barrel-shaped body.

The eye drop applicator system may further include a fluid reservoir with a fluid dispensing portion, wherein the stem includes a coupling portion for receiving the fluid reservoir so that the fluid dispensing portion thereof is oriented transverse to a longitudinal axis of the barrel-shaped body.

The eye drop applicator system may further include the fluid reservoir.

The invention further relates to an eye drop applicator system including a base portion having a barrel-shaped body and a stem, and a fluid directing portion demountably coupled to the base portion and having a drip release portion. The stem may be adapted to retain a fluid reservoir for providing eye drop fluid.

The invention additionally relates to an eye drop applicator system including a base portion having a barrel-shaped body and a stem, a bracket portion slidably associated with the stem, and a fluid directing portion demountably coupled to the base portion and having a drip release portion. The bracket portion is adapted to retain a fluid reservoir in fluid communication with the fluid directing portion.

Also, the invention relates to an eye drop applicator system including a base portion having a barrel-shaped body and a stem, a fluid directing portion demountably coupled to the base portion and having a drip release portion, and a collapsible fluid reservoir in fluid communication with the fluid directing portion.

Furthermore, the invention relates to a method for administering eye drops to an eye including: demountably coupling a fluid reservoir to a fluid directing portion associated with a barrel-shaped body so that a tip of the fluid reservoir communicates with a conduit in the fluid directing portion; rotating the barrel-shaped body against a lower eyelid to retract the eyelid and expose a cul-de-sac of the eye; transmitting fluid from the fluid reservoir to the cul-de-sac through an opening in the fluid directing portion.

The method further may include demountably coupling the fluid directing portion to the barrel-shaped body. The method also may include demountably coupling the fluid reservoir to a stem associated with the barrel-shaped body.

The method may include disposing the barrel-shaped body to abut soft tissue of a central region of the lower eyelid, with the opening in the fluid directing portion oriented away from the eye. In some instances, the opening may be initially oriented away from the eye and the barrel-shaped body may be rotated between about 30° and about 120° to transmit the fluid to the cul-de-sac. In other instances, the opening may be initially oriented away from the eye and the barrel-shaped body may be rotated between about 70° and about 100° to transmit the fluid to the cul-de-sac.

In addition, the present invention relates to a device that applies a simple retracting mechanism to the lower eyelid that opens the cul-de-sac under direct vision (using a mirror if done by the patient for himself). The device may be configured for use with either a multi-dose or a single dose vial. In a preferred exemplary embodiment, the device does not obscure the eye or require the recipient to tilt the head back to any significant degree (i.e. where he might lose visual control) in one exemplary preferred embodiment, the device provides a sterile conduit for the drops to reach the eye with a very soft dispensing tip that may contact the mucous membranes of the lid in a safe manner. Preferably, the device is compact and convenient to carry and use. It places the drop(s) directly into the cul-de-sac of the lower lid thus avoiding splatter, discomfort and misplacement of the medication. In the preferred exemplary embodiment, the device works equally well on either eye, requires minimal dexterity, and may be operated with one hand. The device preferably has no sharp edges and poses less risk to the eye than the dropper alone since it is both softer and has larger radii at the ends.

A removable cover, cap, or case may be included to keep one or more portions of each of the applicator systems of the present invention clean between uses.

### BRIEF DESCRIPTION OF THE DRAWINGS

Preferred features of the present invention are disclosed in the accompanying drawings, wherein:

FIG. 1A shows a perspective view of a first embodiment of an eye drop application system of the present invention;

FIG. 1B shows a top view of the base portion of the system of FIG. 1A;

FIG. 1C shows a perspective view of the base portion of FIG. 1B;

FIG. 1D shows a perspective view of the fluid directing portion of the system of FIG. 1A;

FIG. 1E shows a perspective view of a fluid directing portion connected to a fluid reservoir of the system of FIG. 1A;

FIG. 1F shows a perspective view of another fluid directing portion according to the present invention;

FIG. 1G shows a perspective view of yet another fluid directing portion according to the present invention;

FIG. 1H shows a cross-sectional perspective view taken along a central plane through the longitudinal axis of the fluid directing portion of FIG. 1G;

FIG. 2 shows a perspective view of a second embodiment of an eye drop application system of the present invention;

FIG. 3 shows a perspective view of a third embodiment of an eye drop application system of the present invention;

FIG. 4A shows a perspective view of a fourth embodiment of an eye drop application system of the present invention;

FIG. 4B shows a top view of the system of FIG. 4A;

FIG. 4C shows a perspective view of the bracket portion of the system of FIG. 4A;

FIG. 4D shows a perspective view of the fluid reservoir of the system of FIG. 4A;

FIG. 5A shows a perspective view of another embodiment of an eye drop application system of the present invention (without a fluid reservoir shown);

FIG. 5B shows a bottom perspective view of the system of FIG. 5A;

FIG. 6 shows a perspective view of another embodiment of an eye drop application system of the present invention;

FIG. 7 shows a perspective view of yet another embodiment of an eye drop application system of the present invention;

FIG. 8A shows a top view of another embodiment of an eye drop application system of the present invention;

FIG. 8B shows a back view of the system of FIG. 8A; and

FIG. 8C shows a side view of the system of FIG. 8A.

### DETAILED DESCRIPTION OF THE PREFERRED EMBODIMENTS

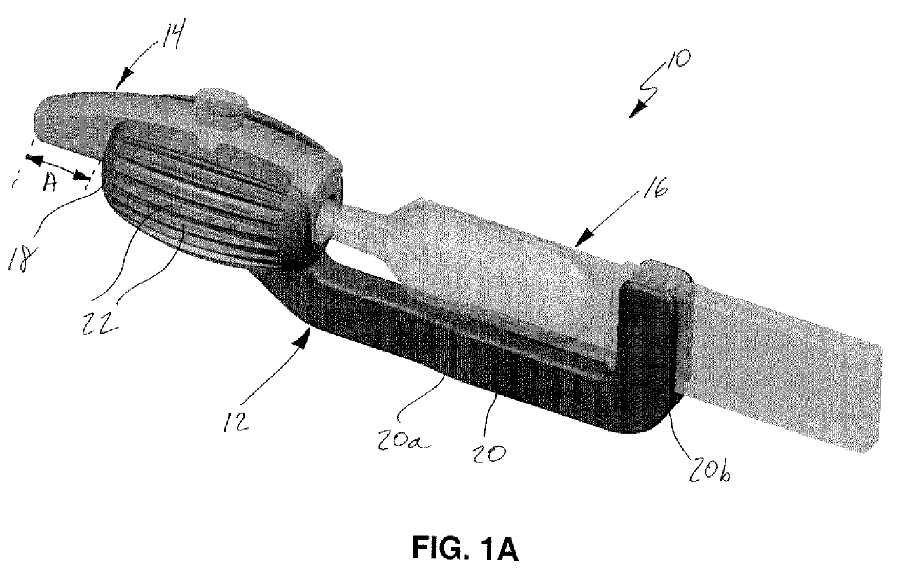

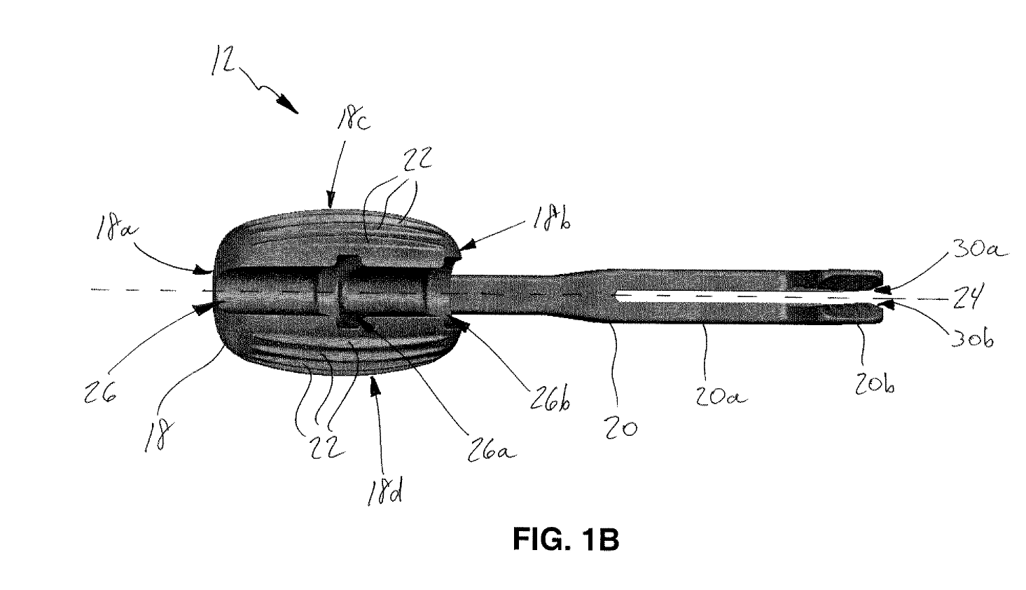

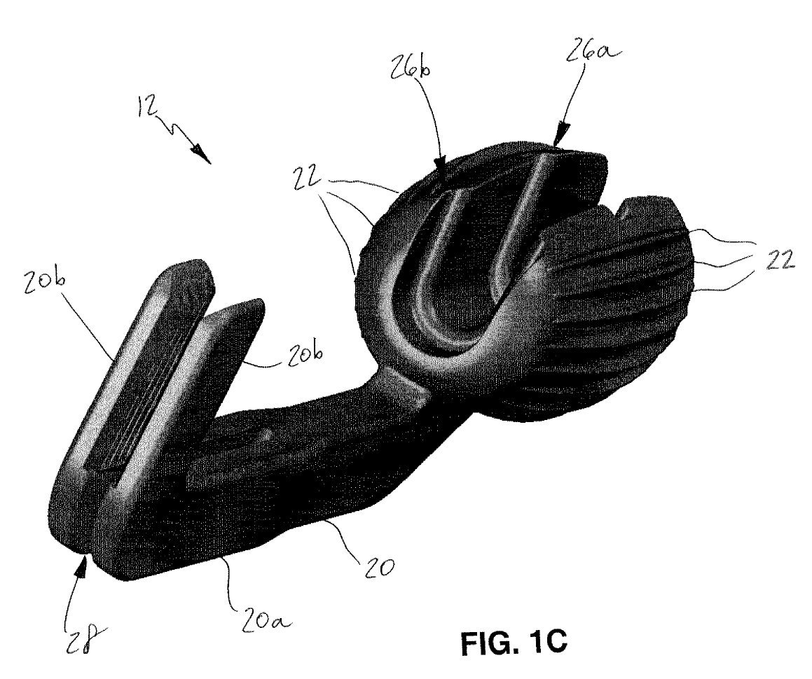

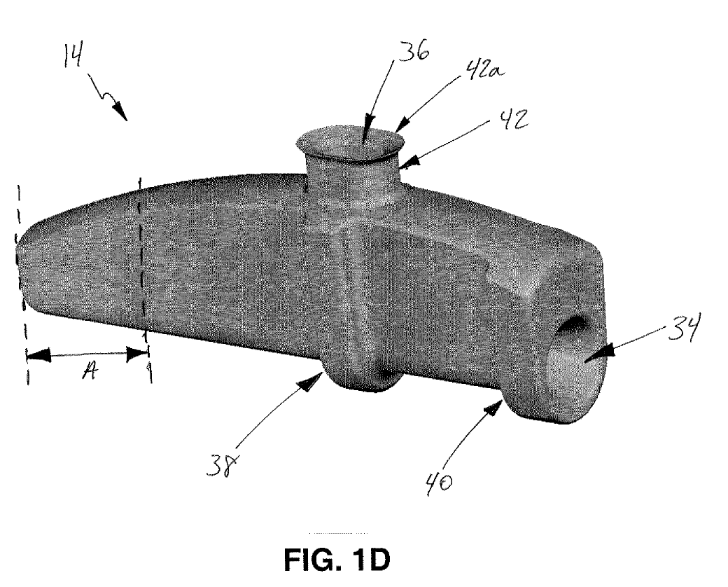

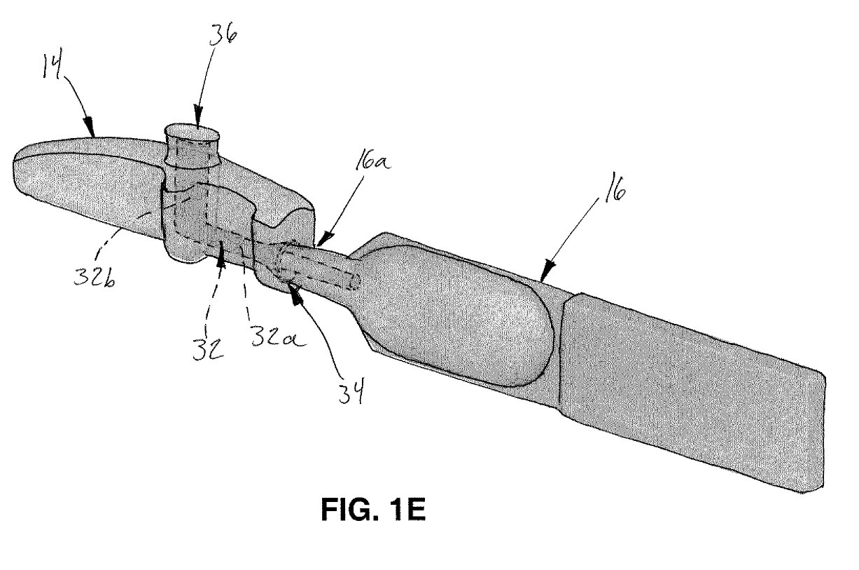

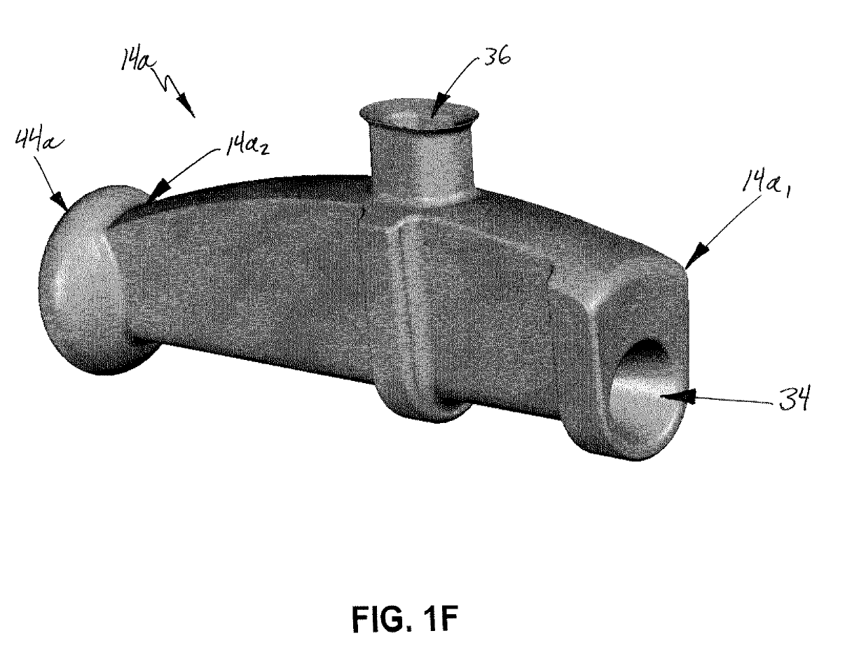

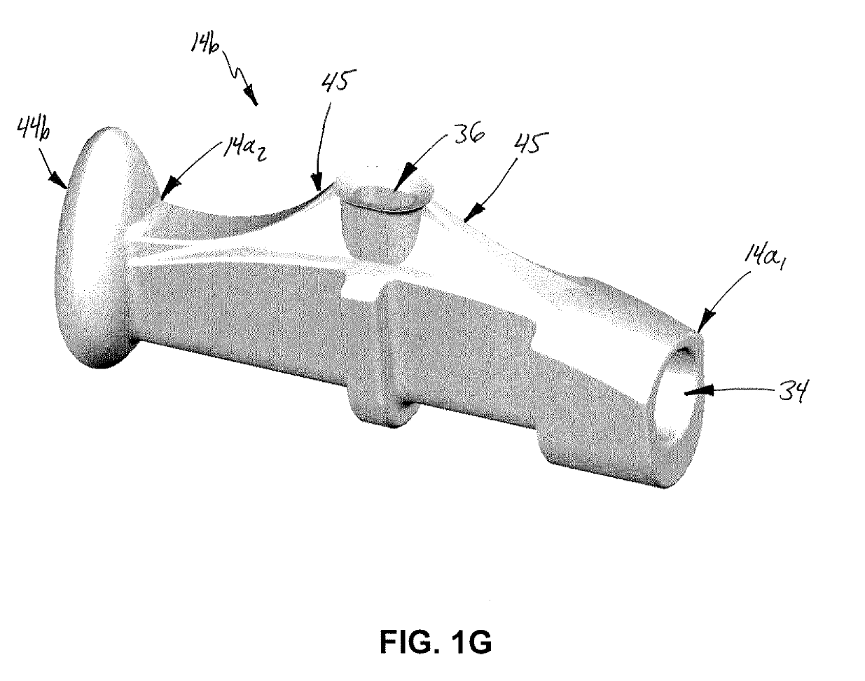

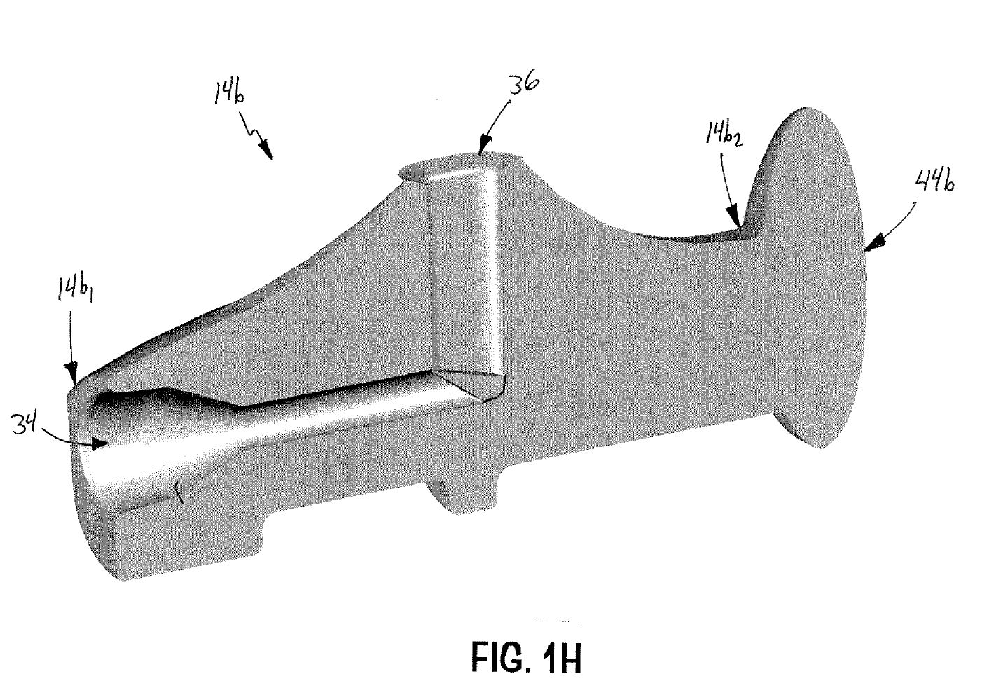

Turning to FIG. 1A, a first embodiment of an eye drop applicator system is shown. Applicator system 10 includes a base portion 12 and a fluid directing portion 14. A fluid reservoir 16 such as a sterile unit dose container of eye drops may be demountably coupled to base portion 12 and connected to fluid directing portion 14 as will be described shortly. Such unit dose containers may be available, for example, from Alcon Corp., Bausch & Lomb, or other manufacturers.

Base portion 12 includes a generally barrel-shaped body 18 coupled to a stem 20. In the preferred exemplary embodiment, body 18 is textured. Preferably, the texturing of body 18 includes longitudinal ridges or ribs 22 that are disposed generally parallel to the longitudinal axis 24 of body 12. Base portion 12 preferably is symmetric about a plane extending perpendicular to the printed page through central axis 24 as shown in FIG. 1B. In one exemplary embodiment, eight ribs extend substantially between ends 18a, 18b of body 18 on each side 18c, 18d.

Body 18 additionally defines a keyway 26 for receiving fluid directing portion 14. In particular, in the exemplary preferred embodiment, keyway 26 defines an opening that includes a generally U-shaped groove 26a and a generally U-shaped, recessed shoulder portion 26b.

In the exemplary preferred embodiment the stem 20 of body 18 includes a first leg 20a and second leg 20b that are disposed generally perpendicular to one another. As shown in FIGS. 1B-1C, a portion of stem 20 preferably is bifurcated with a slot 28 extending therein. The opposing spaced tongs of second leg 20b, created by slot 28, may flex with respect to the plane extending perpendicular to the printed page through central axis 24 thus permitting a fluid reservoir 16 to be releasably captured and retained therein. Opposing surfaces 30a, 30b may be provided with texturing such as serrations to provide additional gripping of reservoir 16.

Turning to FIGS. 1D and 1E, fluid directing portion 14 includes a conduit 32 extending between opening 34 for receiving the fluid dispensing tip of fluid reservoir 16 and opening 36 for dispensing fluid traveling along conduit 32 to an eye. Preferably, when seated in keyway 26 of base portion 12, a first leg 32a of conduit 32 extends parallel to longitudinal axis 24 while a second leg 32b extends generally perpendicular to axis 24. Portion 14 further includes protruding portions 38, 40 for mating with groove 26a and shoulder portion 26b, respectively, of body 18. Thus, portion 14 is configured and dimensioned to be received in keyway 26 of body 18. In some preferred embodiments, portion 14 engages keyway 26 with sufficient friction to provide some resistance to removal therefrom.

Opening 34 may be funnel-shaped to provide a tapered region for receiving the tip of fluid reservoir 16 to create a fluid seal at that juncture. In one preferred exemplary embodiment, fluid directing portion 14 is formed of a generally soft polymer and is single-use and thus disposable. Portion 14 may be provided in pre-packed, individual sterile packs.

At least one of the base portion 12 with its barrel-shaped body 18 and the fluid directing portion 16 may be latex-free.

In the preferred exemplary embodiment, opening 36 is disposed at the end of a protruding drip release portion 42 with an upper rim 42a for facilitating dispensing of eye drops. Preferably, portion 42 provides a soft, flexible exit and drip edge for drop release.

In some embodiments, fluid directing portion 14 may include a region configured and dimensioned to be grasped by a user such as between a thumb and forefinger. As shown for example in FIGS. 1A and 1D, a region A of fluid directing portion 14 is sized to extend outward from barrel-shaped body 18. In one preferred exemplary embodiment, region A is disposed proximate the free end of fluid directing portion 14 opposite the free end that includes opening 34. Region A is sized so that a user need only contact this region when removing fluid directing portion 14 from its packaging and also need only contact this region of fluid directing portion 14 when installing fluid directing portion 14 in keyway 26 in body 18. Thus, contamination of other portions of fluid directing portion 14, including openings 34, 36, may be avoided. Such an extension provided by region A also may function as a locating feature by contacting the side of the bridge of the nose in a manner that helps a user center fluid directing portion 14 below the eye.

A variety of other fluid directing portion designs may be used in accordance with the present invention. For example, as shown in FIG. 1F, fluid directing portion 14a, which is substantially similar to fluid directing portion 14, may include a first end 14a₁ having an opening 34 and a second end 14a₂ having a bulbous portion 44a which for example may be generally disk-like such as circular in perimeter. Bulbous portion 44a for example may have several uses. First, bulbous portion 44a may be grasped or manipulated by a user so that the user need only contact portion 44a when removing fluid directing portion 14a from its packaging and also need only contact this region of fluid directing portion 14a when installing fluid directing portion 14a in keyway 26 in body 18. Thus, contamination of other portions of body 18, including openings 34, 36, may be avoided. Second, bulbous portion 44a may function as a locating feature by contacting the side of the bridge of the nose in a manner that helps a user center fluid directing portion 14a below the eye.

Similarly, as shown in FIGS. 1G-1H, another fluid directing portion 14b, which is similar to fluid directing portion 14, may include a first end 14b₁ having an opening 34 and a second end 14b₂ having an oblong bulbous portion 44b which for example may be oval in perimeter. An arcuate bridge portion 45 also may be included on opposing sides of opening 36. With the exception of the shape and use of bulbous portion 44b and bridge portion 45, the descriptions of fluid directing portions 14, 14a elsewhere herein are applicable to fluid directing portion 14b.

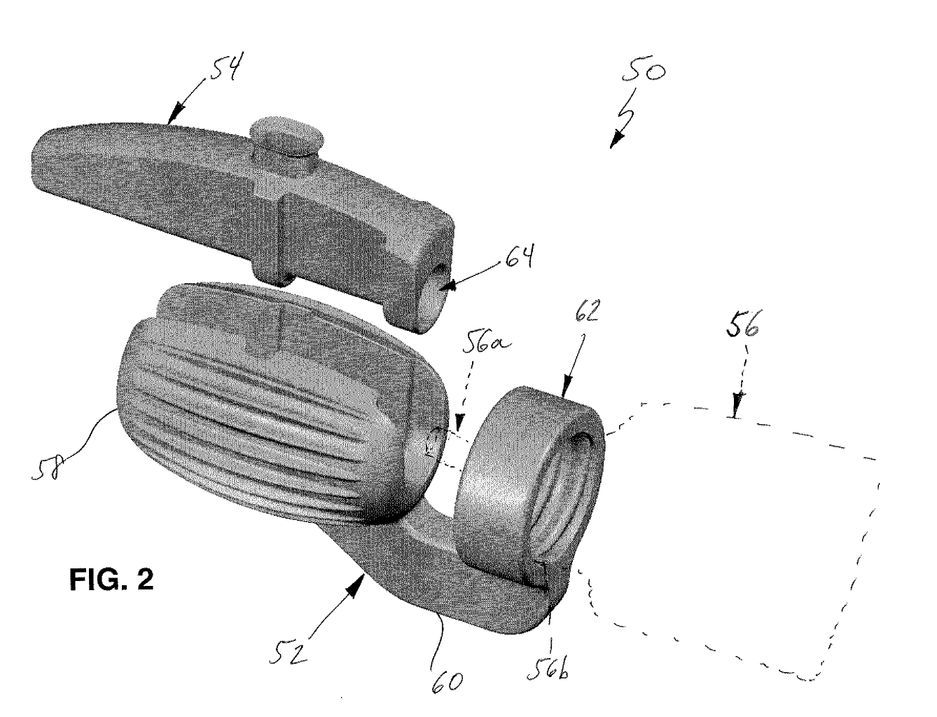

As shown in FIG. 2, a second embodiment of an eye drop applicator system 50 includes a base portion 52 and a fluid directing portion 54. A fluid reservoir 56 such as a multi-dose eye drop bottle may be demountably coupled to base portion 52 and connected to fluid directing portion 54. Body 58 is substantially the same as body 18 described above, while stem 60 is not bifurcated and is shorter than stem 20 described above. The threaded end cap or neck region of the bottle may be threadably associated with internally threaded ring portion 62 of base portion 52. Such eye drop bottles are typically used by manufacturers such as Pfizer, Allergan Inc., Alcon Laboratories Inc., Bausch & Lomb Inc., Advanced Medical Optics Inc., Novartis Pharmaceuticals Inc., and Merck & Co. Inc.

Advantageously, eye drop applicator systems 10, 50 do not leave a tip 16a or 56a of the eye drop fluid reservoir suspended in space, but instead the tip is pressed into a tapered opening 34 or 64 in fluid directing portion 14, 54, respectively.

Any of a variety of neck regions 56b of eye drop fluid reservoirs are contemplated for use with the present invention. For example, although ring portion 62 is shown in FIG. 2 as being internally threaded, ring portion 62 instead may be internally smooth and/or tapered to accommodate and securely receive neck regions 56b of fluid reservoirs 56 that also are smooth and/or tapered. For example, the neck region of the Visine Pure Tears product by Pfizer is not threaded prior to the fluid dispensing tip.

The operation of systems 10, 50 will now be described, particularly with reference to system 10 for convenience. However, the use of the features of system 10 as described herein also apply to the features of system 50.

The longitudinal ribs 22 and associated indentations of body 18 advantageously facilitate frictional gripping when ribs 22 contact the lower lid and peri-orbital skin of a user. The bulk of the barrel-shaped body 18 and the added friction of its irregular surface combine to allow retraction and displacement of the central region of the lower eyelid and local peri-orbital soft tissues as body 18 is manually rotated and depressed into the soft tissue to open the cul-de-sac. The curved shape of body 18 encourages greater movement of the more mobile central part of the eyelid.

In practice, a new sterile fluid directing portion 14 is placed for each application of eye drops and then removed and discarded. Alternatively, a removable cover, cap, or case may be used to keep one or more portions of each of the applicator systems of the present invention clean between uses such as a cover to be disposed over fluid directing portion 14 and barrel-shaped body 18. Portion 14 may be inserted bare handed without contacting sterile surfaces that guide the eye drop fluid or touch the mucous membranes of the eyelid by grasping region A between a thumb and forefinger for removal from packaging and placement into slot 26 of body 18. Portion 14 is thus inserted into body 18 of base portion 12 and the fluid reservoir 16 is secured to base portion 12 so that a fluid seal is achieved at the junction of the dispensing tip of the fluid reservoir 16 and opening 34 of portion 14. Then, fluid reservoir 16 is squeezed until a drop begins to form at the drip release portion 42 that is held upright initially. The body 18 (particularly the ribs 22 thereof) is then brought into contact with the lower eyelid and tilted/rotated for example by about 90° and depressed into the peri-orbital soft tissues to pull the lower eyelid down and open the cul-de-sac. As body 18 is rotated, the drop in drip release portion 42 is released into the cul-de-sac. Release of a drop may be facilitated by squeezing fluid reservoir 16 to release fluid therefrom and allowing gravity to move the drop from opening 36 to an eye initially spaced at a distance from the drop. Alternatively, a drop resting on upper rim 42a of protruding drip release portion 42 may be allowed to contact the fluid of the eye causing transfer of the drop from portion 42 to the eye via surface tension.

In some embodiments, opening 36 may be initially oriented away from the eye and the barrel-shaped body 18 may be rotated between about 30° and about 120° to transmit the fluid to the cul-de-sac. In other instances, opening 36 may be initially oriented away from the eye and the barrel-shaped body 18 may be rotated between about 70° and about 10° to transmit the fluid to the cul-de-sac.

In one preferred exemplary embodiment, the semi-rigid body 18 and stem 20 are made of a relatively stiff injection molded plastic that may be wiped clean and reused for multiple applications of eye drop medication. In this embodiment, the fluid directing portion 14 preferably is supplied in a sterile state, packed in an individual container, and opened and coupled to body 18 only when fluid is to be administered to an eve. Fluid directing portion 14 may be injection molded using a very soft, chemically inert, biocompatible elastomer such as silicone.

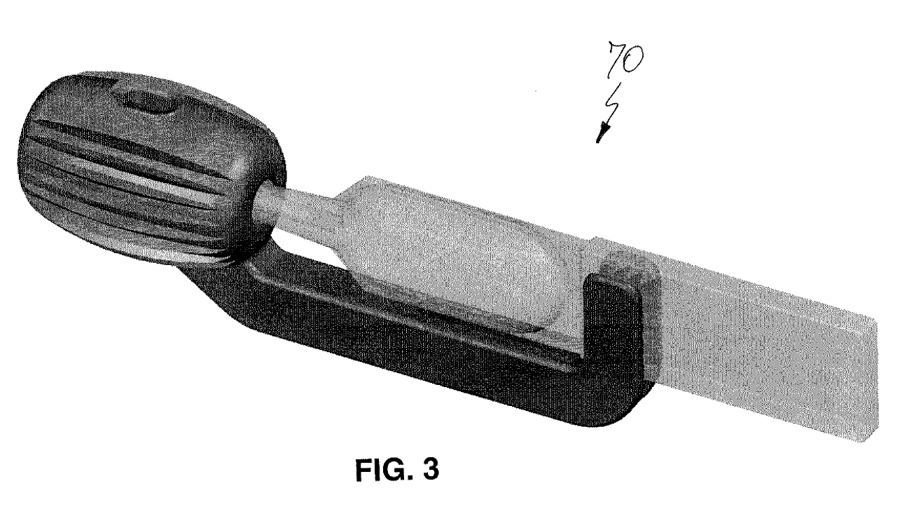

In an alternate embodiment, when sterility is not required and merely clean is sufficient, base portion 12 and a fluid directing portion 14 may be integrally formed and thus made as a single part that may be washable and re-usable (and/or alternatively may be placed in a removable cover, cap, or case to keep one or more portions of each of the applicator systems of the present invention clean between uses). An exemplary embodiment of such an eye drop applicator system 70 is shown in FIG. 3, for which the structure and operation is otherwise the same as disclosed above with respect to systems 10, 50.

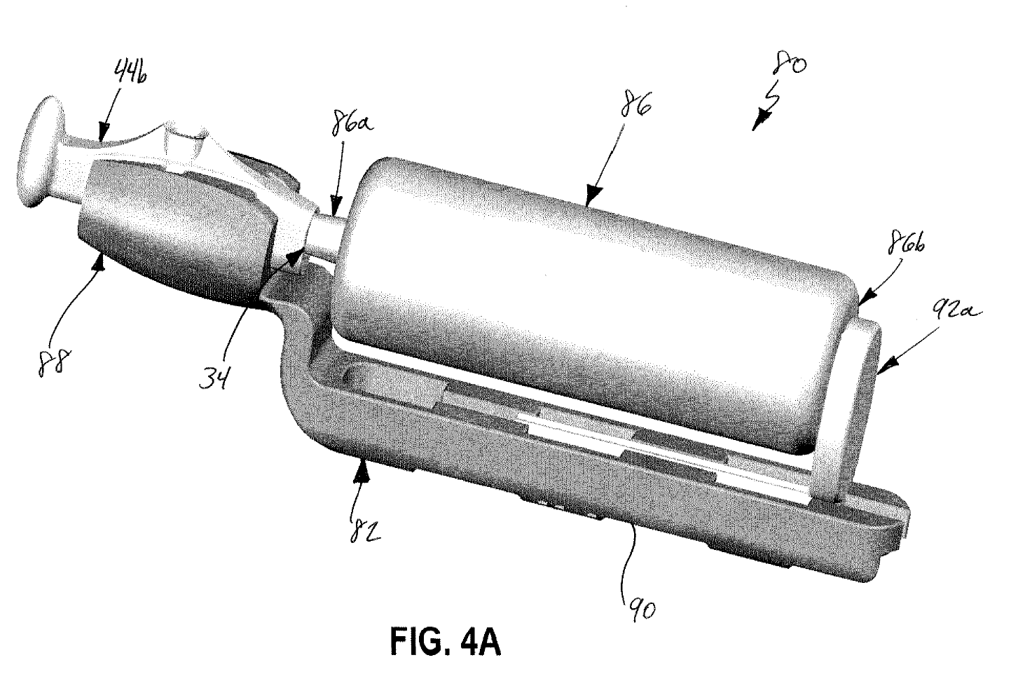

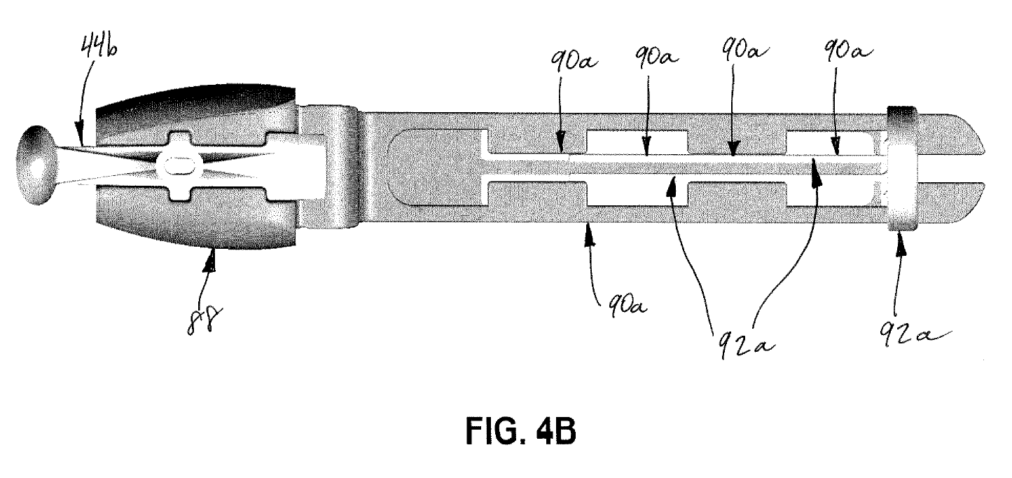

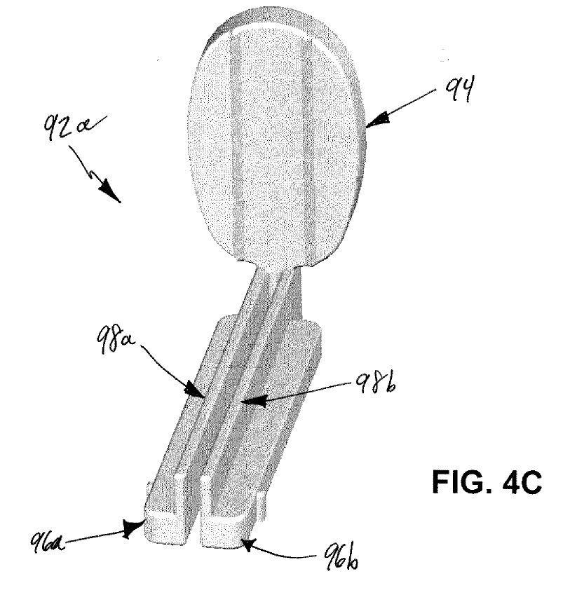

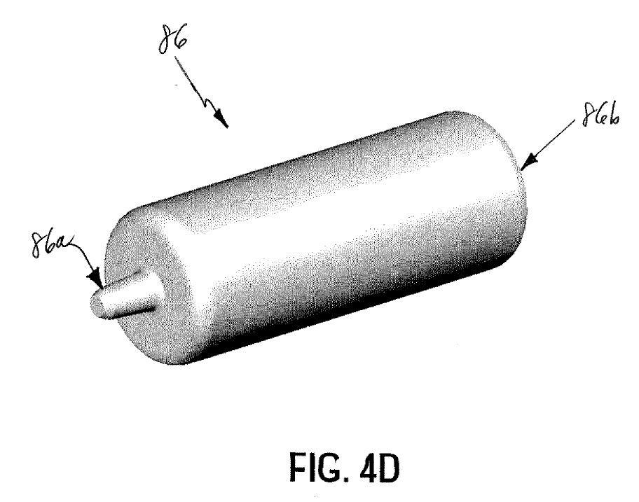

Turning to FIGS. 4A-D, another embodiment of an eye drop applicator system according to the present invention is shown. Applicator system 80 includes a base portion 82 and for example a fluid directing portion 44b (although the other fluid directing portions disclosed herein also may be used). A fluid reservoir 86 with tip 86a, such as a sterile container of eye drops, may be demountably coupled to base portion 82 and connected to fluid directing portion 44b.

Base portion 82 includes a generally barrel-shaped body 88 coupled to a stem 90. In the preferred exemplary embodiment, body 88 is configured and textured as previously described for example with respect to body 18.

A movable bracket portion 92a is received and moves along a slotted portion or track 90a formed in stem 90. Referring to FIG. 4C, bracket portion 92a includes a plate 94 (which for example may be generally oval in perimeter shape) for abutting a free end of a fluid reservoir 86 opposite the end with tip 86a. Bracket portion 92 further includes a pair of spaced side portions 96a, 96b and a pair of rails 98a, 98b which in the preferred exemplary embodiment are disposed substantially perpendicular to portions 96a, 96b. Thus, rails 98a, 98b move with respect to track 90a so that reservoir 86 may be securely retained with tip 86a disposed in opening 34 and plate 92a abutting a free end 86b of reservoir 86 opposite tip 86a.

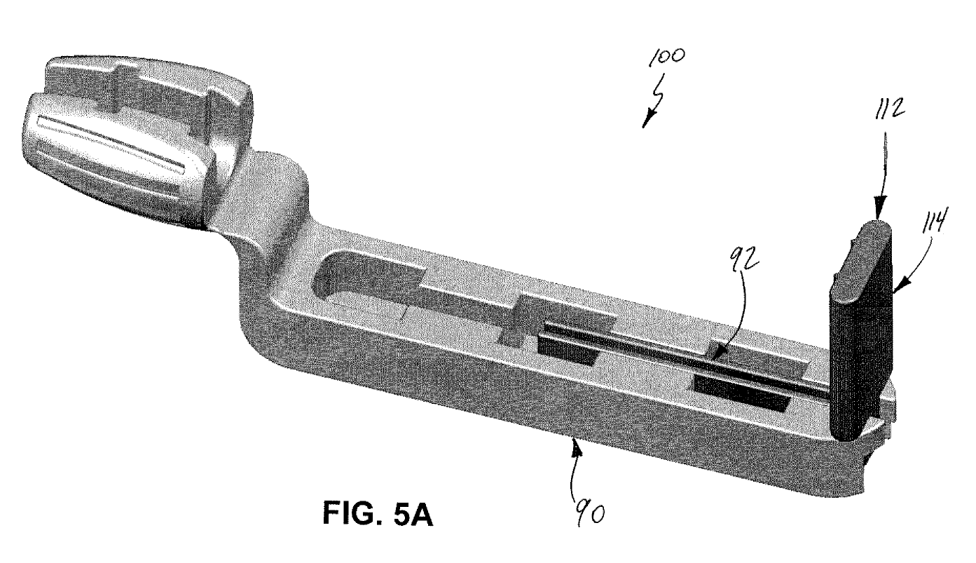

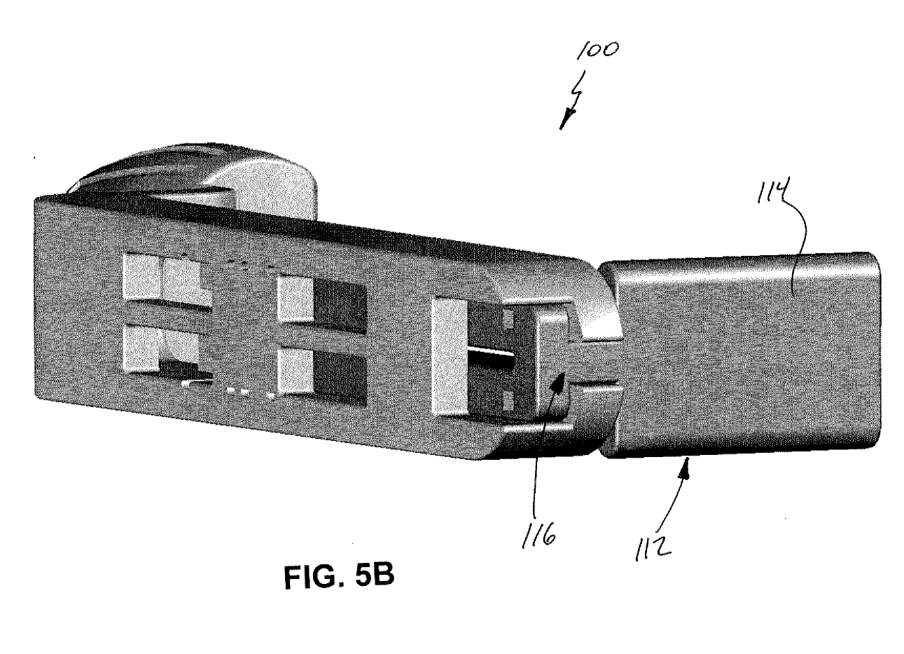

Referring to FIGS. 5A-5B, another embodiment of an eye drop applicator system according to the present invention is shown, similar to applicator system 80 of FIGS. 4A-D. Applicator system 100 is largely as described above with respect to other embodiments. However, bracket portion 112 includes a plate 114 having a generally rectangular shape connected to a generally T-shaped portion 116. Stem 90 includes a slot 92 configured and dimensioned to receive T-shaped portion 116 so that bracket portion 112 is linearly movable with respect to stem 90 about a central longitudinal axis thereof.

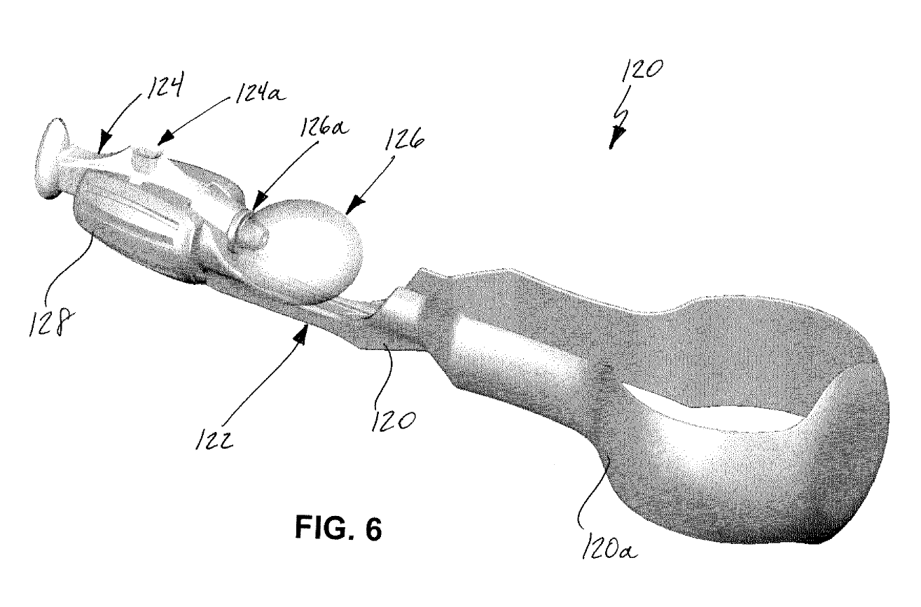

Turning to FIG. 6, yet another embodiment of an eye drop applicator system according to the present invention is shown. Applicator system 120 includes a base portion 122 and a fluid directing portion 124. A fluid reservoir 126 such as a squeezable reservoir of eye drops that for example may be generally spherical and/or collapsible as fluid is gradually dispensed therefrom, may be demountably coupled to fluid directing portion 124. Base portion 122 includes a generally barrel-shaped body 128 coupled to a stem 120, which may also include a handle portion 120a.

It is known that many currently available multi-dose dropper bottles are 25 less than half-full of fluid when new. Such currently available and are designed to deliver drops by minimal squeezing with the dropper tip fully dependent (i.e. pointed down), so that the fluid therein can be dropped without air mixing with the fluid and can readily fall off the tip into the eye. Because of the dependent orientation of the dropper, the fact that the bottle may be filled with air by 50% or more by volume does not substantially effect the effectiveness of the dropper.

In some of the embodiment of the present inventions such as shown in FIGS. 1A, 2, 3, and 4A, the fluid reservoir (which may be a dropper bottle) is held generally horizontally, in line with the eyelid roller/retractor barrel with its fluid directing portion. In such designs, unless the fluid reservoirs are easily squeezable and/or generally full of fluid, difficulties can be encountered in discharging the fluid from the reservoir.

The embodiment of FIG. 6, for example, uses a collapsible fluid reservoir 126 that for example may be filled by a user with fluid from a multi-dose bottle. In one exemplary preferred embodiment, reservoir 126 is sized to hold the entire contents of a multi-dose vial (i.e., up to about 1 oz.). In other embodiments, reservoir 126 may be filled with smaller volumes of fluid such as about 0.1-0.3 oz. In a preferred exemplary embodiment, reservoir 126 is generally spherical in shape and has an integral valve 126a that resists fluid leakage. Reservoir 126 preferably has a single opening for filling and delivering fluid. Preferably, fluid is readily discharged from reservoir 126 by squeezing between two fingers such as the thumb and index finger once mounted on base portion 122 and fluidly communicating with fluid directing portion 124. And, reservoir 126 preferably will contain little if any air when filled and/or as it is emptied of fluid during repeated use.

In the exemplary embodiment, reservoir 126 will be sterilized before filling with fluid and may be connected in a sterile manner to or be integral with fluid directing portion 124. In some exemplary embodiments, fluid reservoir 126 is made of a transparent injection molded material such as silicone, or it may be a color coded translucent material to indicate a type of medication fluid contained therein. Reservoir 126 preferably is designed to be filled by a user from many types of multi-dose dropper bottles, or, alternatively, it may be designed so that it can be filled by a pharmaceutical company for prolonged shelf life (and serve as an alternative to a traditional multi-dose dropper bottle).

As shown in FIG. 6, system 120 has a generally rigid structure with three main portions that support the fluid storage and transfer components and provide for manual control. This structure is similar to that used for a unit dose dropper and includes the following: a barrel-shaped body 128 is for retracting the lower eyelid and opening the lower cul-de-sac of the eye and accepts the fluid directing portion 124 with drop release opening 124a; a central area is provided for receiving and maintaining the position of a fluid reservoir 126; and a handle portion 120a is provided to allow secure one-handed control and axial rotation of applicator system 120 using the palm and fingers other than those being used to squeeze the reservoir during drop delivery. The handle size and shape shown in FIG. 6 is only one preferred embodiment for use with the present invention.

Preferably, reservoir 126 can be easily filled by a user for example from a multi-dose bottle and includes at least one valve, cap, or other stopper that will maintain reservoir 126 in a fluid-tight state after filling and between uses. One or more such valves, for example, may be formed during injection molding of reservoir 126 and/or fluid directing portion 124.

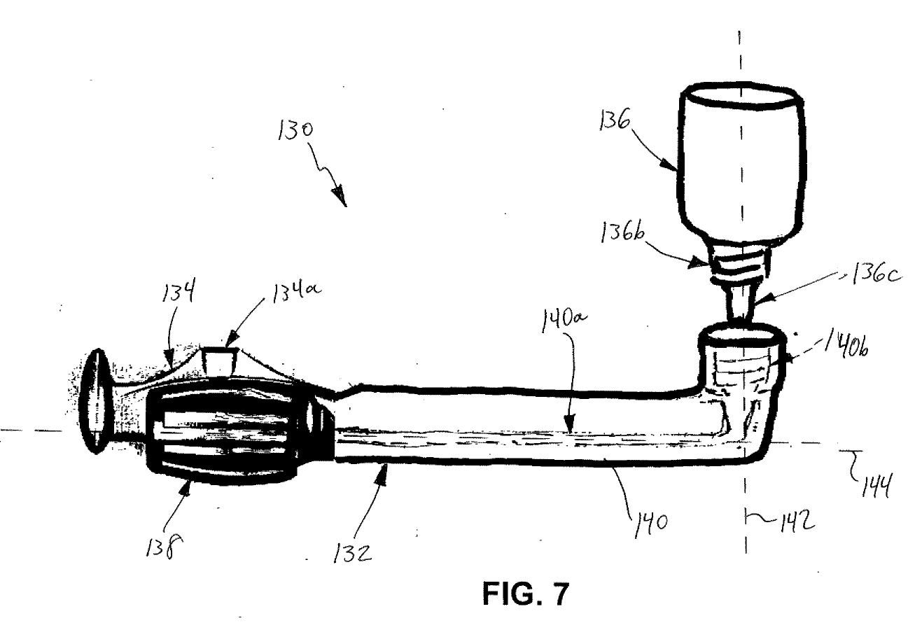

Turning next to FIG. 7, an alternate embodiment of the configuration of eye drop applicator system for example of FIG. 2 is shown. In FIG. 7, applicator system 130 includes a base portion 132, a fluid directing portion 134, and a fluid reservoir 136, with base portion 132 including a barrel-shaped body 138 coupled to a stem 140 having a fluid conduit 140a integrally formed therein. Threaded portion 136b of reservoir 136, for example, may be threadably associated with internally threaded portion 140b of stem 140. As shown in FIG. 7, reservoir 136 may be oriented transverse to, and preferably substantially perpendicular to fluid conduit 140a. In particular, reservoir 136 may be dispose on an axis 142 while fluid conduit 140a may be disposed along an axis 144, with these axes 142, 144, being transverse to one another and preferably perpendicular to one another. Such an orientation, with tip 136c oriented generally downward, may facilitate transfer of fluid from reservoir 136 ultimately to opening 134a of fluid directing portion 134 (which is in fluid communication with fluid conduit 140a). As discussed previously, such an orientation of reservoir 136 also may advantageously minimize problems associated with air present along with the drop fluid in reservoir 136. It should be noted that tip 136c of reservoir 136 is oriented to deliver fluid opposite to the direction that fluid exits opening 134a of fluid directing portion 134. Thus, fluid may be dispensed from reservoir 136, be transferred to fluid directing portion 134, and subsequently delivered to a user's eye through opening 134a of fluid directing portion 134. It also should be noted that although fluid directing portion 134 preferably is spaced from reservoir 136 to permit sufficient separation so that reservoir 136 may be spaced from a user's head during the eye dropping procedure, stem 140 may be a variety of lengths to suit a given need.

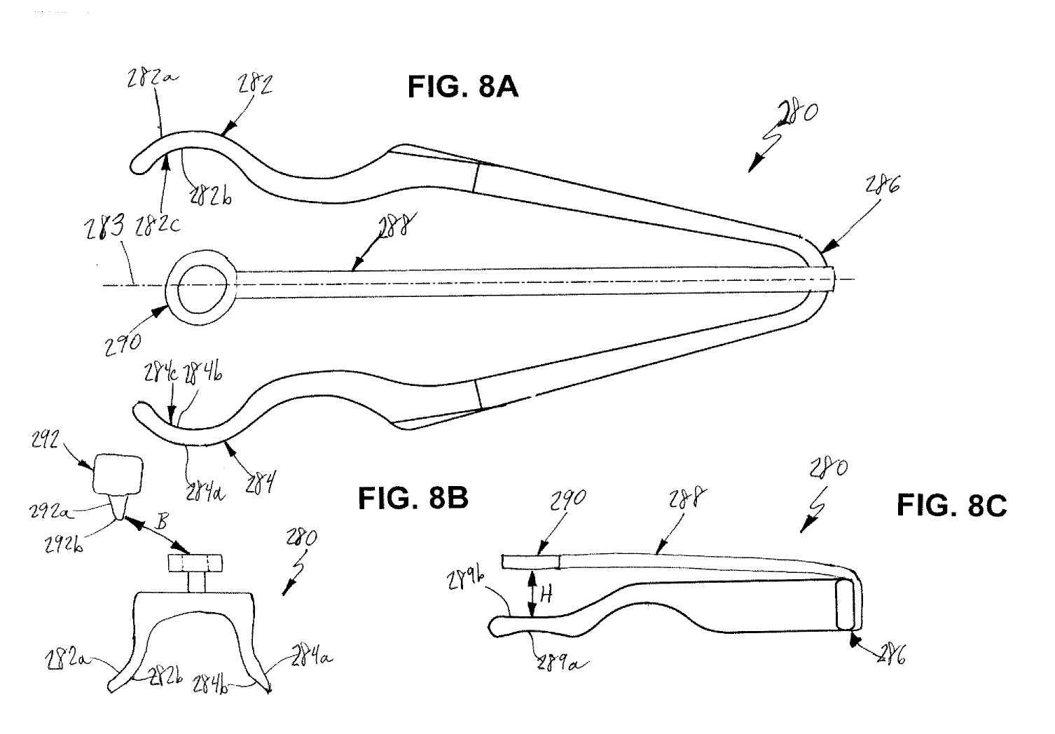

Turning to FIGS. 8A-8C, another embodiment of an eye drop delivery system 280 is shown. In particular, system 280 includes a first arm 282, a second arm 284, and a joint region 286 disposed between and coupling arms 282, 284. First arm 282 is configured and dimensioned to accommodate the furrow of an upper eyelid and second arm 284 is configured and dimensioned to accommodate the furrow of a lower eyelid. Arms 282, 284 are so configured and dimensioned that, when in use, they contact only the outer skin of the eyelid without contacting any mucous membrane or inside of the eye. Thus, eye contamination may be avoided. Arm 282 has outer and inner edges 282a, 282b, respectively, while arm 284 has outer and inner edges 284a, 284b, respectively. Preferably, at least a portion of each of inner edges 282b, 284b is beveled along eyelid contacting portions 282c, 284c.

In the preferred exemplary embodiment, an extension 288 extends from either of arms 282, 284 or joint region 286 (as shown) so that retaining portion 290 proximate a free end thereof is disposed intermediate arms 282, 284 and along centerline 283. Portion 290 also is spaced a height H from arms 282, 284 on the side of system 280 opposite the side that includes lower edge 289 which is placed against a patient's eyelids.

In one preferred exemplary embodiment, retaining portion 290 is spaced from upper edge 289b such that when the neck 292a of an eye drop fluid reservoir 292 is secured by retaining portion 290, the tip 292b will not extend to the plane through upper edge 289b proximate eyelid contacting portions 282c, 284c. In other words, tip 292b is to be held spaced from a user's eyeball when arms 282, 284 are applied to the outer skins of the eyelids.

For example, the upward movement of first arm 282 would retract the upper eyelid upward due to the friction and adhesion between the arm 282 and the outer skin of the upper eyelid. Similarly, the downward movement of the second arm 284 would retract the lower eyelid downward due to the friction and adhesion between arm 284 and the outer skin of the lower eyelid. These movements achieve the retraction of the eyelids. In some circumstances when retraction is desired for only one of the upper or lower eyelids, only one arm 282, 284 is applied to the respective eyelid to be retracted. Regardless, in the exemplary embodiment, tip 292b of reservoir 292 may be demountably coupled to retaining portion 290 before retraction so that fluid release from tip 292b is directed to the eye without the need for a user to separately grasp and aim reservoir 292.

As shown in FIG. 8A, in the preferred exemplary embodiment, retaining portion 290 is ring shaped and also may be tapered to accommodate a tapered neck of a reservoir 292 introduced for example as shown by arrow B. In alternate embodiments, retaining portion 290 may be in the form of a clamp or other securing region that for example may snap-fit or otherwise retain a portion of reservoir 292 such as neck 292a.

In some exemplary embodiments, retaining portion 290 is angulated so that fluid from reservoir 290 is directed toward the cul-de-sac of the eye exposed by the retracting action.

U.S. Pat. No. 6,544,169 B2 to Putrino et at., which discloses eyelid retraction devices that may be used and modified in connection with the present invention, is incorporated herein in its entirety by reference thereto.

While various descriptions of the present invention are described above, it should be understood that the various features can be used singly or in any combination thereof. Therefore, this invention is not to be limited to only the specifically preferred embodiments depicted herein. Further, it should be understood that variations and modifications within the spirit and scope of the invention may occur to those skilled in the art to which the invention pertains. For example, the applicator systems disclosed herein may be adapted for use with all types of eye medications, including liquids, salves, and ointments. Thus, a variety of types of functionalities of fluid reservoir tips such as tips 16a, 56a and a variety of functionalities of drip release portions such as portion 42 are contemplated in accordance with the present invention, including dispensing mechanisms for liquids, salves, and ointments in the form, for example, of a spray. In addition, although not shown, a removable cover, cap, or case may be used to keep one or more portions of each of the applicator systems of the present invention clean between uses. Moreover, various portions of each of the applicator systems of the present invention may be integrally formed. For example, with respect to applicator system 10 of FIG. 1A, two or more of base portion 12, fluid directing portion 14, and fluid reservoir 16 may be integrally formed. Accordingly, all expedient modifications readily attainable by one versed in the art from the disclosure set forth herein that are within the scope and spirit of the present invention are to be included as further embodiments of the present invention. The scope of the present invention is accordingly defined as set forth in the appended claims.

What is claimed is:

1. An eye drop applicator system comprising:
a barrel-shaped body;
a stem; and
a fluid directing portion;
wherein the stem is adapted to retain a fluid reservoir for providing eye drop fluid.

2. The eye drop applicator system of claim 1, wherein the barrel-shaped body and stem are integrally formed.

3. The eye drop applicator system of claim 1, wherein the barrel shaped body and fluid directing portion are integrally formed.

4. The eye drop applicator system of claim 1, wherein the fluid directing portion is demountably coupled to the barrel-shaped body.

5. The eye drop applicator system of claim 4, wherein the barrel-shaped body comprises a keyway that receives a like-shaped protruding region on the fluid directing portion.

6. The eye drop applicator system of claim 5, wherein the fluid directing portion is sized to extend along a longitudinal axis of the barrel-shaped body beyond the barrel-shaped body to form a free end of the eye drop applicator system.

7. The eye drop applicator system of claim 6, wherein the free end comprises a bulbous portion.

8. The eye drop applicator system of claim 1, wherein the fluid directing portion comprises a first opening for receiving fluid from the fluid reservoir and a second opening for dispensing fluid from the fluid reservoir.

9. The eye drop applicator system of claim 8, wherein the fluid directing portion comprises a conduit extending from the first opening to the second opening.

10. The eye drop applicator system of claim 8, wherein the conduit comprises first and second sections disposed substantially perpendicular to one another.

11. The eye drop applicator system of claim 1, wherein the fluid directing portion comprises a drip release portion proximate an opening thereof.

12. The eye drop applicator system of claim 1, wherein the barrel-shaped body comprises texturing.

13. The eye drop applicator system of claim 12, wherein the barrel-shaped body is disposed along a longitudinal axis and the texturing comprises ribs disposed substantially parallel to the longitudinal axis.

14. The eye drop applicator system of claim 1, wherein the stem is bifurcated.

15. The eye drop applicator system of claim 1, wherein the stem extends substantially parallel to a longitudinal axis of the barrel-shaped body.

16. The eye drop applicator system of claim 1, wherein the stem comprises a clamping portion for releasably retaining the fluid reservoir.

17. The eye drop applicator system of claim 1, wherein the stem comprises a ring-shaped portion for receiving a neck of the fluid reservoir.

18. The eye drop applicator system of claim 17, wherein the ring-shaped portion is threaded for threadably receiving the neck of the fluid reservoir.

19. The eye drop applicator system of claim 1, wherein at least one selected from the group consisting of the barrel-shaped body and the fluid directing portion is latex-free.

20. The eye drop applicator system of claim 1, wherein the stem comprises a coupling portion for receiving a fluid reservoir with a tip so that the tip is oriented transverse to a longitudinal axis of the barrel-shaped body.

21. The eye drop applicator system of claim 20, wherein the tip is oriented substantially perpendicular to the longitudinal axis of the barrel-shaped body.

22. The eye drop applicator system of claim 1, further comprising a fluid reservoir with a fluid dispensing portion, wherein the stem comprises a coupling portion for receiving the fluid reservoir so that the fluid dispensing portion thereof is oriented transverse to a longitudinal axis of the barrel-shaped body.

23. The eye drop applicator system of claim 1, further comprising the fluid reservoir.

24. An eye drop applicator system comprising:
a base portion having a barrel-shaped body and a stem; and
a fluid directing portion demountably coupled to the base portion and having a drip release portion;
wherein the stem is adapted to retain a fluid reservoir for providing eye drop fluid.

25. An eye drop applicator system comprising:
a base portion having a barrel-shaped body and a stem;
a bracket portion slidably associated with the stem; and
a fluid directing portion demountably coupled to the base portion and having a drip release portion;
wherein the bracket portion is adapted to retain a fluid reservoir in fluid communication with the fluid directing portion.

26. An eye drop applicator system comprising:
a base portion having a barrel-shaped body and a stem;
a fluid directing portion demountably coupled to the base portion and having a drip release portion; and
a collapsible fluid reservoir in fluid communication with the fluid directing portion.

27. A method for administering eye drops to an eye comprising:
demountably coupling a fluid reservoir to a fluid directing portion associated with a barrel-shaped body so that a tip of the fluid reservoir communicates with a conduit in the fluid directing portion;
rotating the barrel-shaped body against a lower eyelid to retract the eyelid and expose a cul-de-sac of the eye;
transmitting fluid from the fluid reservoir to the cul-de-sac through an opening in the fluid directing portion.

28. The method of claim 27, further comprising:
demountably coupling the fluid directing portion to the barrel-shaped body.

29. The method of claim 27, further comprising:
demountably coupling the fluid reservoir to a stem associated with the barrel-shaped body.

30. The method of claim 27, further comprising:
disposing the barrel-shaped body to abut soft tissue of a central region of the lower eyelid, with the opening in the fluid directing portion oriented away from the eye.

31. The method of claim 30, wherein the opening is initially oriented away from the eye and the barrel-shaped body is rotated between about 30° and about 120° to transmit the fluid to the cul-de-sac.

32. The method of claim 30, wherein the opening is initially oriented away from the eye and the barrel-shaped body is rotated between about 70° and about 100° to transmit the fluid to the cul-de-sac.
